(19) United States
(12) Patent Application Publication
(10) Pub. No.: US 2019/0216437 A1
(43) Pub. Date: Jul. 18, 2019

(54) Ultrasonic Diagnostic Apparatus and Image Processing Method

(71) Applicant: Hitachi, Ltd., Chiyoda-ku, Tokyo (JP)

(72) Inventor: Tetsuya YAMADA, Chiyoda-ku, Tokyo (JP)

(21) Appl. No.: 16/335,783

(22) PCT Filed: Dec. 8, 2017
(86) PCT No.: PCT/JP2017/044143

§ 371 Date: Mar. 22, 2019

(30) Foreign Application Priority Data
Mar. 21, 2017 (JP) 2017-054752

(51) Int. Cl. A61B 8/08; G06T 5/00; G06T 5/50; A61B 8/14

(52) U.S. Cl. CPC A61B 8/5246; G06T 5/009; G06T 5/50; A61B 8/5207; G06T 2207/30104; A61B 8/488; G06T 2207/10072; G06T 2207/20208; G06T 2207/20221; A61B 8/14

(57) ABSTRACT

A correction coefficient k is generated based on a combination of a luminance value I constituting a tomographic image F1 and a power value P constituting a power image F2. The power value P is suppressed by multiplying the power value P by the correction coefficient k. After such pre-processing, any one of the color data set (R1, G1, B1) corresponding to the luminance value I and the color data set (R2, G2, B2) corresponding to the power value P (after suppression) is selected.

### TECHNICAL FIELD

The present invention relates to an ultrasonic diagnostic apparatus and an image processing method, and particularly to synthesis of a plurality of ultrasonic images.

### BACKGROUND ART

The ultrasonic diagnostic apparatus is a medical apparatus that forms an ultrasonic image by transmitting and receiving ultrasonic waves to and from a living body, and processing a received signal obtained by this manner. The ultrasonic diagnostic apparatus may generate a first ultrasonic image and a second ultrasonic image at the same time, and the first ultrasonic image and the second ultrasonic image may be synthesized to obtain a synthesized image which may be displayed. For example, the first ultrasonic image is a tomographic image as a monochrome image representing a cross section of a tissue, and the second ultrasonic image is a power image as a color image representing a two-dimensional distribution of the power of Doppler information on the cross section. In this case, the tomographic image is a tissue image, and the power image is a blood flow image.

Several image synthesis methods are known. A first method is a selection method or a superimposition method, in which any one of pixel values is selected from a first pixel value constituting the first ultrasonic image and a second pixel value constituting the second ultrasonic image for each display coordinate (pixel) (see Patent Literature 1). A second method is a blending method, in which a new pixel value is generated by performing blending processing on the first pixel value and the second pixel value for each display coordinate (see Patent Literature 2 and Patent Literature 3).

### PRIOR ART LITERATURE

### Patent Literature

PTL 1: JP-A-2001-269344

PTL 2: JP-A-2004-135934

PTL 3: JP-A-2006-55241

### SUMMARY OF INVENTION

### Technical Problem

In a case where a tomographic image and a power image are synthesized, a problem in which the color power image is excessively displayed is pointed out. For example, it has been pointed out that, after the synthesis, a color portion may be superimposed and displayed on a tissue boundary, or a color portion may extend beyond the inside of a blood vessel to a blood vessel wall. That is, a color image representing blood flow is superimposed and displayed at a place where blood flow should not exist. Particularly, in a case where a luminance value and a power value are compared and any one of the values is selected from the comparison result, the above problem is likely to occur. This problem can also occur in other cases where a monochrome image and a color image are synthesized.

An object of the invention is to prevent any of the images from being excessively displayed in a case where a synthesized image is generated by synthesizing the first ultrasonic image and the second ultrasonic image. Alternatively, an object of the invention is to prevent a power image from being unnecessarily imaged in a case where a tomographic image and a power image are synthesized. Alternatively, an object of the invention is to solve or alleviate a problem that easily occurs in a method in which any one of two pixel values is selected for each display coordinate when the method is adopted.

### Solution to Problem

An ultrasonic diagnostic apparatus according to an embodiment includes a pre-processing section that performs pre-processing on an input pixel value pair including a first input pixel value constituting a first ultrasonic image and a second input pixel value constituting a second ultrasonic image, in which a correction coefficient is generated based on at least one input pixel value in the input pixel value pair, and at least one input pixel value in the input pixel value pair is corrected based on the correction coefficient; and a synthesis section that inputs an input pixel value pair after pre-processing, generates an output pixel value constituting a display image based on the input pixel value pair after pre-processing, and outputs the output pixel value.

According to the above configuration, one or both of the input pixel values in the input pixel value pair input to the synthesis section are pre-processed by the pre-processing section prior to the input. The pre-processing corrects at least one of the two input pixel values based on at least one of the two input pixel values. For example, in the two input pixel values, one input pixel value tends to occur excessive display is suppressed before synthesis processing. Alternatively, it is conceivable to enhance the other input pixel value before the synthesis processing. The input pixel value is a concept including a color data set (for example, a set of value R, value G and value B) generated by conversion. Similarly, the output pixel value is a concept including a color data set as well.

In the synthesis section, the above configuration effectively functions in a case where two input pixel values are mutually compared and anyone of the input pixel values is selected as the output pixel value based on a mutual comparison result. In such a selection method, for example, if the second input pixel value is relatively large with respect to the first input pixel value, the second input pixel value is selected regardless of the specific values of the first input pixel value and the second input pixel value. In contrast, according to the above configuration, since one of the two input pixel values to be mutually compared is corrected (for example, the second input pixel value is suppressed), it is possible to prevent or reduce the occurrence of a problem occurring under the processing condition without changing the processing condition in the synthesis section. In the above selection method, it is generally required to maintain the input pixel value since any one of input pixel values is output as an output pixel value as it is. However, in an aspect that no particular problem occurs even if the input pixel value is corrected, it is possible and appropriate to adopt the above pre-processing.

In the embodiment, the first ultrasonic image is a tomographic image representing a cross section of a tissue; the second ultrasonic image is a power image representing a two-dimensional distribution of power of Doppler information; the display image is a synthesized image generated by synthesizing the tomographic image and the power image; the first input pixel value is a luminance value corresponding to an echo value; and the second input pixel value is a power value. For example, in a case where the input pixel value is a value illustrating speed, elasticity or the like, an observation value or a diagnosis value to be read is changed by correcting the input pixel value. In contrast, in a case where the input pixel value is the luminance value or the power value, even if the input pixel value is corrected, no particular problem occurs in the observation of the ultrasonic image. As described above, it is desirable to apply the above configuration to the combination of the tomographic image and the power image. However, the first ultrasonic image may be a monochrome image other than the tomographic image, and the second ultrasonic image may be a color image other than the power image.

In the embodiment, the pre-processing section includes a generation section that generates the correction coefficient based on at least the luminance value, and a correction section that corrects the power value based on the correction coefficient, and the correction coefficient functions as a coefficient that suppresses the power value. The correction coefficient is generated by referring to the luminance value, and the power value is suppressed based on the correction coefficient, so that the problem that the power image is excessively displayed is solved or alleviated. In the embodiment, the generation section generates the correction coefficient based on a combination of the luminance value and the power value. According to the configuration, since the degree of suppression of the power value is adaptively determined according to the combination of the two input pixel values, the excessive display of the power image can be more appropriately and naturally suppressed.

In the embodiment, the synthesis section is a section that selects any one of the luminance value and the power value after correction based on a mutual comparison between the luminance value and the power value after correction, and the luminance value tends to be selected as a result of the mutual comparison when the power value is suppressed, and in a case where the power value after correction is selected as the output pixel value, the output pixel value is suppressed. In the above configuration, on a premise of the selection method, a selection condition (in some cases, also the output pixel value) is operated by correcting the input pixel value. The power value reflects the Doppler information of the blood flow, which, however, also changes due to an angle of the ultrasonic beam, tissue property and the like. There is basically no problem even if the power value itself is corrected since the power image inherently illustrates a rough flow of blood flow or an existence range thereof.

An image processing method according to the embodiment includes a step of performing pre-processing on an input pixel value pair including a first input pixel value constituting a first ultrasonic image and a second input pixel value constituting a second ultrasonic image, that is, correcting at least one input pixel value in the input pixel value pair based on at least the other input pixel value in the input pixel value pair; and a step of inputting the input pixel value pair after pre-processing, selecting any one of the input pixel values based on mutual comparison of the input pixel value pair after pre-processing, and outputting the selected input pixel value as an output pixel value.

In the above configuration, on a premise of the selection method based on the mutual comparison, at least the other input pixel value is corrected based on at least one input pixel value prior to the mutual comparison. According to the above configuration, even if the selection method based on the mutual comparison is maintained as it is, the selection condition or the selection result thereof can be changed according to the situation. The above method may be implemented as a function of hardware or as a function of software. In the latter case, a program for executing the method may be installed on the ultrasonic diagnostic apparatus via a storage medium or via a network.

In the embodiment, the first ultrasonic image is a monochrome tomographic image representing a tissue, the second ultrasonic image is a color power image representing blood flow, and a power value which is the second input pixel value is corrected before the selection step is executed. The pre-processing may be selectively executed by a user. According to the configuration, it is possible to selectively display the display image generated after the execution of the pre-processing and the display image generated without executing the pre-processing.

### BRIEF DESCRIPTION OF DRAWINGS

FIG. 1 is a block diagram illustrating an ultrasonic diagnostic apparatus according to an embodiment.

FIG. 2 is a conceptual diagram illustrating a basic function of a display processing unit illustrated in FIG. 1.

FIG. 3 is a block diagram illustrating a first example of an image processing method (image synthesis method).

FIG. 4 is a conceptual diagram illustrating a function of a correction coefficient generator illustrated in FIG. 3 as a three-dimensional function.

FIG. 5 is a diagram illustrating several cross sections of the three-dimensional function illustrated in FIG. 4.

FIG. 6 is a diagram illustrating a display image before the image processing method is applied and a display image after the display image is applied.

FIG. 7 is a block diagram illustrating a second example of the image processing method.

FIG. 8 is a block diagram illustrating a third example of the image processing method.

FIG. 9 is a block diagram illustrating a fourth example of the image processing method.

FIG. 10 is a block diagram illustrating a fifth example of the image processing method.

FIG. 11 is a block diagram illustrating a sixth example of the image processing method.

FIG. 12 is a conceptual diagram illustrating a function of a correction coefficient generator illustrated in FIG. 11 as a three-dimensional function.

FIG. 13 is a diagram illustrating several cross sections of the three-dimensional function illustrated in FIG. 12.

### DESCRIPTION OF EMBODIMENTS

Hereinafter, an embodiment will be described with reference to the drawings.

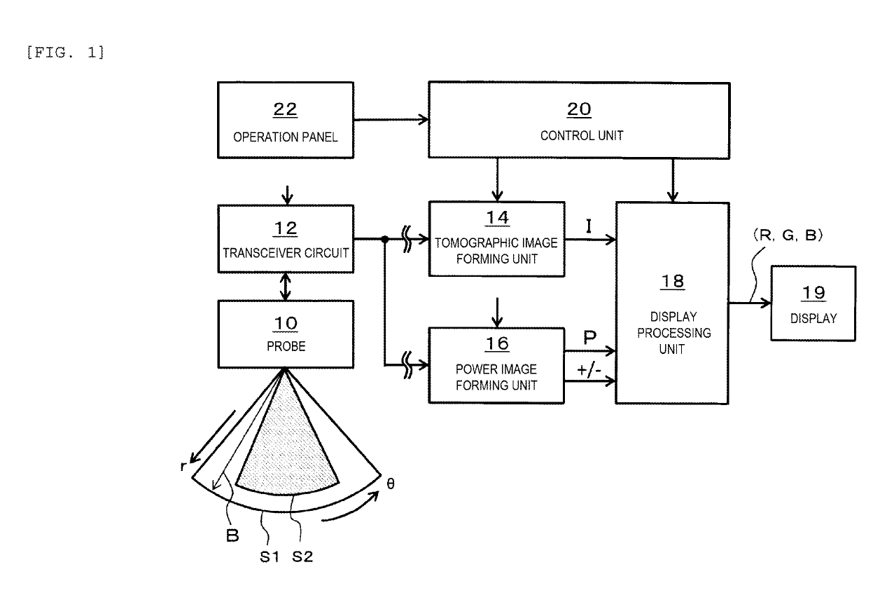

An ultrasonic diagnostic apparatus according to an embodiment is illustrated as a block diagram in FIG. 1. The ultrasonic diagnostic apparatus is an apparatus that is installed in a medical institution such as a hospital and forms and displays an ultrasonic image based on a received signal obtained by transmitting and receiving ultrasonic waves to and from a living body. In the present embodiment, a tomographic image representing a cross section of a tissue and a power image illustrating a two-dimensional distribution of the power of the Doppler information on the cross section are formed as the ultrasonic image, and a synthesized image obtained therefrom is displayed. The tomographic image is a monochrome image, which may also be referred to as a tissue image. The power image is a color image, which may also be referred to as a blood flow image. In the power image, the blood flow flowing in a positive direction and the blood flow flowing in a negative direction may be separately expressed in different colors, or the blood flow may be expressed in a fixed color regardless of the direction of the flow. The power image is inherently an image expressing the power of Doppler information from the blood flow as a moving body. However, due to various reasons such as movement of the tissue and low distance resolution in a depth direction, the power may be observed and displayed at a site other than the blood flow. A technique for solving or alleviating the problem will be described below.

In FIG. 1, a probe 10 is configured by a probe head, a cable, and a connector. The connector is detachably attached to the ultrasonic diagnostic apparatus body. The probe head is brought into contact with, for example, a surface of an object. In the illustrated example, the probe head includes an array transducer including a plurality of resonator elements arranged one-dimensionally. The array transducer forms an ultrasonic beam B with which an electronic scanning is performed. Abeam scanning plane S1 is formed by the electronic scanning. The beam scanning plane S1 is a two-dimensional echo data acquisition area corresponding to the cross section of the tissue. Abeam scanning plane S2 is formed by the electronic scanning of the ultrasonic beam B or the electronic scanning of another ultrasonic beam. The beam scanning plane S2 is a two-dimensional echo data acquisition area for acquiring Doppler information. The beam scanning plane S2 is usually a part of the beam scanning plane S1. A spread range of the beam scanning plane S2 coincides with a spread range of an area of interest set for power observation. In FIG. 1, r illustrates a depth direction, and e illustrates an electronic scanning direction. Instead of a 1D array transducer, a 2D array transducer may be provided to obtain volume data from a three-dimensional space in the living body. As an electronic scanning method, an electronic sector scanning method, an electronic linear method, and the like are known.

A transceiver circuit 12 is an electronic circuit that functions as a transmission beam former and a reception beam former. At the time of transmission, a plurality of transmission signals is supplied in parallel from the transceiver circuit 12 to the array transducer. As a result, a transmission beam is formed. At the time of reception, reflected waves from the living body are received by the array transducer. As a result, a plurality of received signals is output in parallel from the array transducer to the transceiver circuit 12. The transceiver circuit 12 includes a plurality of amplifiers, a plurality of A/D converters, a plurality of delay circuits, an addition circuit of the like. In the transceiver circuit 12, a plurality of received signals is subjected to phasing addition (delay addition) to form beam data corresponding to the received beam. The reception frame data is configured by a plurality of beam data arranged in the electronic scanning direction. Each beam data is configured by a plurality of echo data arranged in the depth direction.

A tomographic image forming unit 14 functions as a tomographic image forming section, which is an electronic circuit that generates tomographic image data based on the reception frame data. The electronic circuit includes one or a plurality of processors. The tomographic image forming unit 14 includes, for example, a detection circuit, a logarithmic conversion circuit, a frame correlation circuit, and a digital scan converter (DSC). The tomographic image is configured by a plurality of pixel values. Each pixel value is a luminance value I as an echo value. A series of luminance values I are sequentially sent to a display processing unit 18 in a display coordinate order.

A power image forming unit 16 functions as a power image forming section, which is an electronic circuit that generates a power image based on the reception frame data. The electronic circuit includes one or a plurality of processors. The power image forming unit 16 includes a quadrature detection circuit, a clutter removing circuit, an autocorrelation circuit, a speed calculation circuit, a power calculation circuit, and a DSC. The power image is configured by a plurality of pixel values. Each pixel value is a power value P. The power value P is accompanied by a positive or a negative sign (+/−) in the illustrated configuration example. A series of power values P are sequentially sent to the display processing unit 18 in the display coordinate order.

The display processing unit 18 is configured by an electronic circuit including one or a plurality of processors. The display processing unit 18 functions as a pre-processing section, a color conversion section, and a synthesis section. That is, the display processing unit 18 executes a pre-processing step, a color conversion step, and a synthesis step. The pre-processing section includes a correction coefficient generation section and a correction section, and the pre-processing step includes a correction coefficient generation step and a correction step. The synthesis section includes a relative comparison section and a selection section, and the synthesis step includes a relative comparison step and a selection step. The display processing unit 18 synthesizes a tomographic image as a monochrome image and a power image as a color image, thereby generating a synthesized image. The synthesized image is displayed on a display 19 as a display image.

In the present embodiment, at the time of image synthesis, any one of two input pixel values is selected on a display coordinate unit basis. That is, a selection method is adopted instead of a blending method. This will be described in detail later. The display unit 19 is configured by an LCD, an organic EL device or the like.

A control unit 20 functions as a control section that controls each configuration illustrated in FIG. 1, and includes a CPU and an operation program. The control unit 20 may be configured by another programmable processor. An operation panel 22 is connected to the control unit 20. The operation panel 22 includes various input devices such as a trackball, a switch, and a keyboard.

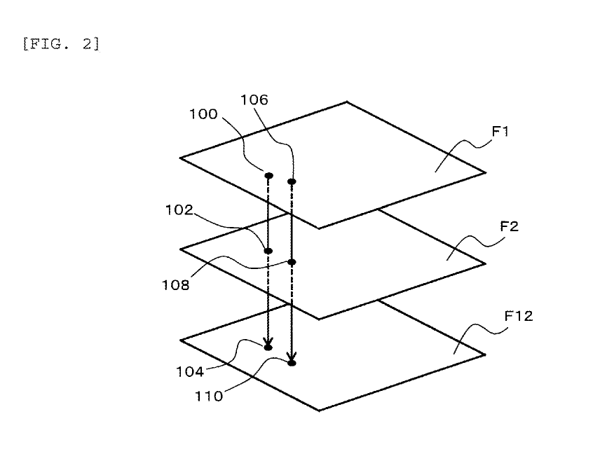

Image synthesis is conceptually illustrated in FIG. 2. In the present embodiment, a tomographic image F1 and a power image F2 are synthesized to generate a synthesized image F12. Specifically, in a case where attention is paid to a first coordinate, two pixel values (input pixel value pairs) 100, 102 at the same first coordinate are compared with each other, any one of the pixel values 100, 102 is selected based on the comparison result, and the selected pixel value is set as a pixel value 104 constituting the synthesized image F12. Similarly, in a case where attention is paid to a second coordinate, two pixel values (input pixel value pairs) 106, 108 at the same second coordinate are compared with each other, any one of the pixel values 106, 108 is selected based on the comparison result, and the selected pixel value is set as a pixel value 110 constituting the synthesized image F12. In the above selection, for example, a larger one of the two pixel values is selected. Alternatively, color data is compared for each color in two color data sets corresponding to the two pixel values, and any one of the color data sets is selected based on the comparison result. The concept of the mutual comparison between the two pixel values includes the mutual comparison between the two color data sets. The concept of selecting any one of the pixel values includes selecting any one of the color data sets.

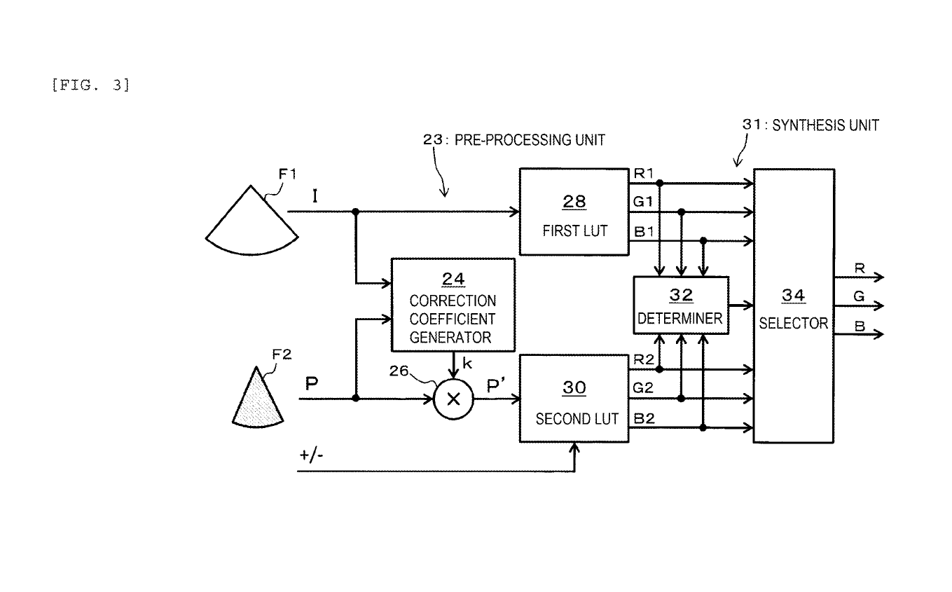

A first configuration example of the display processing unit illustrated in FIG. 1 is illustrated in FIG. 3. The display processing unit includes a pre-processing unit 23 and a synthesis unit 31, and further includes a color conversion unit in the illustrated configuration example. The luminance value I and the power value P are input to the display processing unit as two input pixel values associated with the same coordinates. A correction coefficient generator 24 is configured by a look-up table (LUT) or the like, which generates a correction coefficient k based on a combination of the luminance value I and the power value P. The correction coefficient k is multiplied by the power value P in a multiplier 26. The correction coefficient k may take a value in a range of 0.0 to 1.0 in the configuration example illustrated in FIG. 3. In a case where the power value P is multiplied by a correction coefficient of 1.0, the power value P is substantially preserved. In a case where the power value P is multiplied by a correction coefficient less than 1.0, the power value P is suppressed. This suppression provides two meanings. Firstly, when the power value is suppressed, a possibility that the power value is selected in a determiner 32 to be described later is reduced. Secondly, even in a case where the power value is selected as an output pixel value, the power value becomes smaller by the suppression by the correction coefficient k, and therefore, the pixel corresponding to the power value becomes inconspicuous on the display image.

A first LUT 28 and a second LUT 30 constitute a color conversion unit. In the first LUT 28, a color data set (R1, G1, B1) corresponding to the luminance value I is generated based on the luminance value I. The color data set (R1, G1, B1) is a pixel value as a configuration element of a monochrome image. For example, a minimum echo value is expressed in black, and a maximum echo value is expressed in white. An intermediate echo value is expressed in gray. In the second LUT 30, a color data set (R2, G2, B2) is generated based on a power value P′ after correction and a sign. The color data set (R2, G2, B2) is a pixel value as a configuration element of a color image. For example, the flow in the positive direction and the flow in the negative direction are expressed by separate colors (red and blue). The luminance of each color represents the magnitude of the power. The magnitude of the power may be expressed by luminance of a color such as orange, regardless of the direction of the flow.

In the illustrated configuration example, the determiner 32 selects a pixel value based on the following relationships (1) to (3). Specifically, in a case where the following relationship (1) is satisfied, the power value is selected according to the following relationship (2), and in a case where the following relationship (1) is not satisfied, the luminance value is selected according to the following relationship (3). The selection conditions illustrated below are examples.

(R1<R2) or (G1<G2) or (B1<B2) (1)

OUT(R2,G2,B2) (2)

OUT(R1,G1,B1) (3)

A selector 34 outputs any one of the color data set (R1, G1, B1) and the color data set (R2, G2, B2) in accordance with the selection result. The determiner 32 and the selector 34 may be configured by a single processor.

In the first configuration example illustrated in FIG. 3, the power value P is suppressed based on the combination of the luminance value and the power value prior to the pixel value selection as the synthesis processing. Therefore, even if the synthesis processing conditions are maintained, the synthesis processing conditions can be modified or partially modified as a result of the pre-processing. Particularly, it is possible to solve or reduce a problem that the power image is spread and expressed excessively by suppressing the power value P. In other words, in a case where the selection method is adopted, since an output target is alternatively determined, depending on the situation, there is a tendency that a displayed content becomes excessive or biased. However, the above problem can be improved by appropriately determining the correction coefficient as long as the pre-processing above is combined. In other words, the relationship (3) itself is effective for superimposing and displaying a power image on a tomographic image, but depending on the situation, the power image may be excessively displayed. Such a problem can be solved or alleviated by the pre-processing described above.

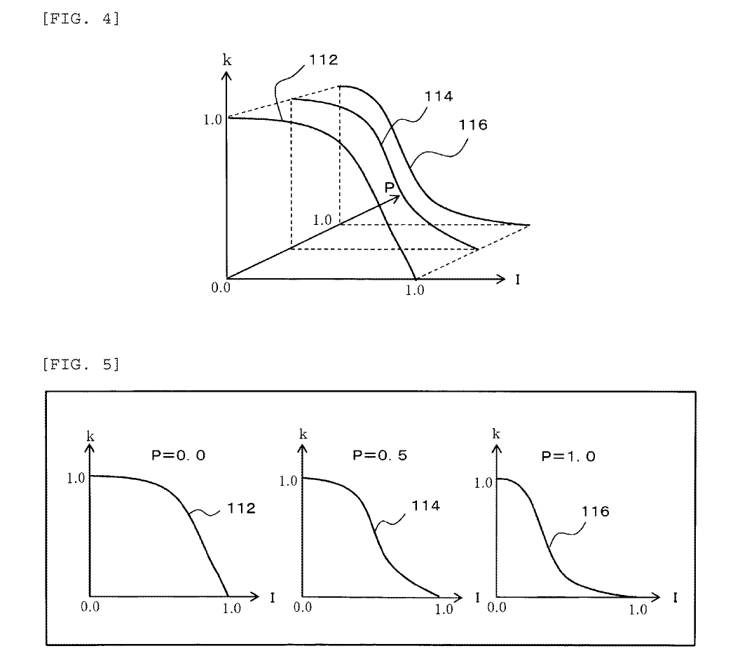

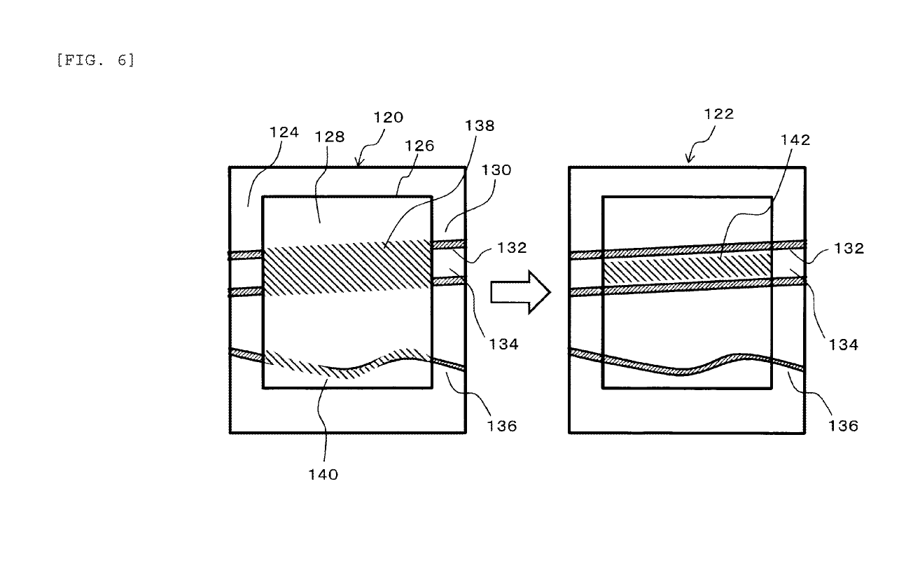

A function of the correction coefficient generator illustrated in FIG. 3 is illustrated as a three-dimensional function in FIG. 4. A first horizontal axis represents a luminance value (however, a normalized luminance value) I. A second horizontal axis represents a power value (however, a normalized power value) P. A vertical axis represents a correction coefficient k. In order to aid in understanding the shape of the three-dimensional function, FIG. 4 illustrates three two-dimensional functions 112, 114, and 116 corresponding to three points of a power value P=0.0, a power value P=0.5, and a power value P=1.0, and contents of which are further illustrated in FIG. 5.

In the illustrated example, in any one of the power values P, the correction coefficient k decreases as the luminance value I increases. As the power value P increases, a falling position is shifted to a lower luminance side in the two-dimensional functions 112, 114, and 116. However, the three-dimensional functions illustrated in FIGS. 4 and 5 are examples.

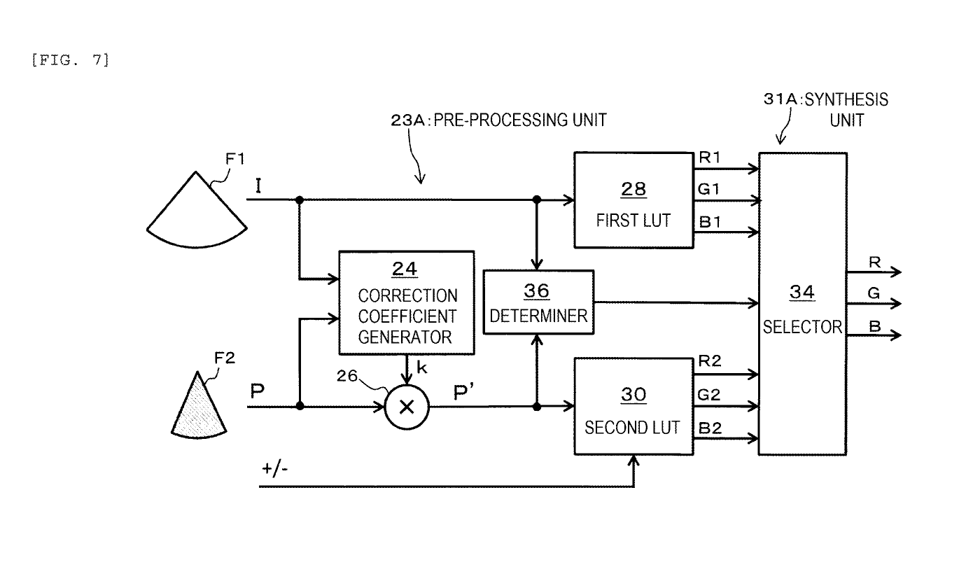

A synthesized image 120 generated without applying the pre-processing according to the present embodiment and a synthesized image 122 generated by applying the pre-processing is schematically illustrated in FIG. 6. The synthesized image 120 is generated by synthesizing a tomographic image and a power image. An ROI 126 determines a display area of a power image 128. The tomographic image includes a cross section of a blood vessel 130, which includes a vessel wall 132 and a lumen (blood flow) 134. Further, the illustrated example includes a tissue boundary portion 136. A power image portion 138 expressed by color extends beyond the lumen 134 of the blood vessel 130 to the vessel wall 132. Such excessive display may occur at a portion where luminance of the tissue is low and a certain degree of power is observed. Further, a power image portion 140 is also superimposed on the tissue boundary portion 136. For example, such a condition may occur when the tissue boundary portion 136 is in a respiratory movement. A result of applying the pre-processing without changing the synthesis processing conditions is illustrated as the synthesized image 122. A power image portion 142 does not extend to the vessel wall 132 and remains within the lumen 134. In addition, the power image portion is not superimposed on the tissue boundary portion 136. As described above, according to the image processing of the present embodiment, it is possible to improve the synthesis processing result and to obtain natural image content while maintaining the synthesis processing conditions.

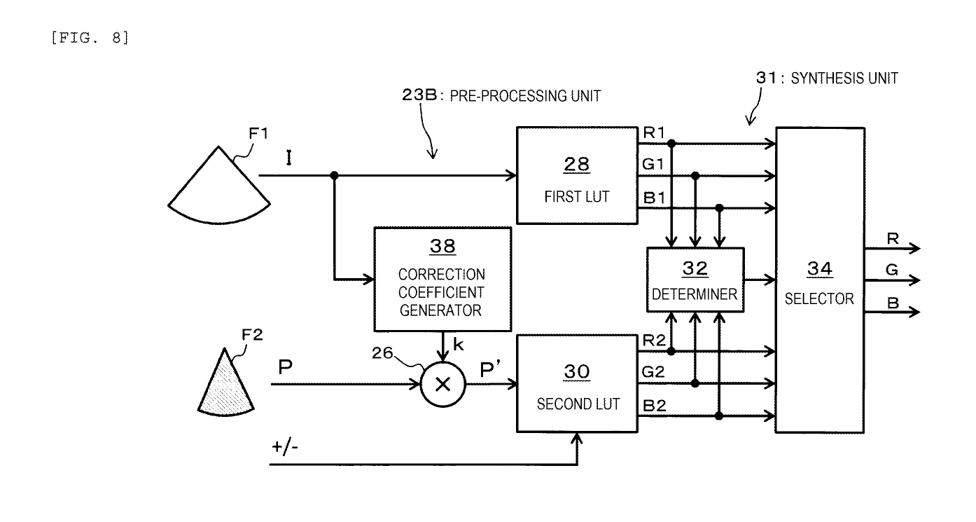

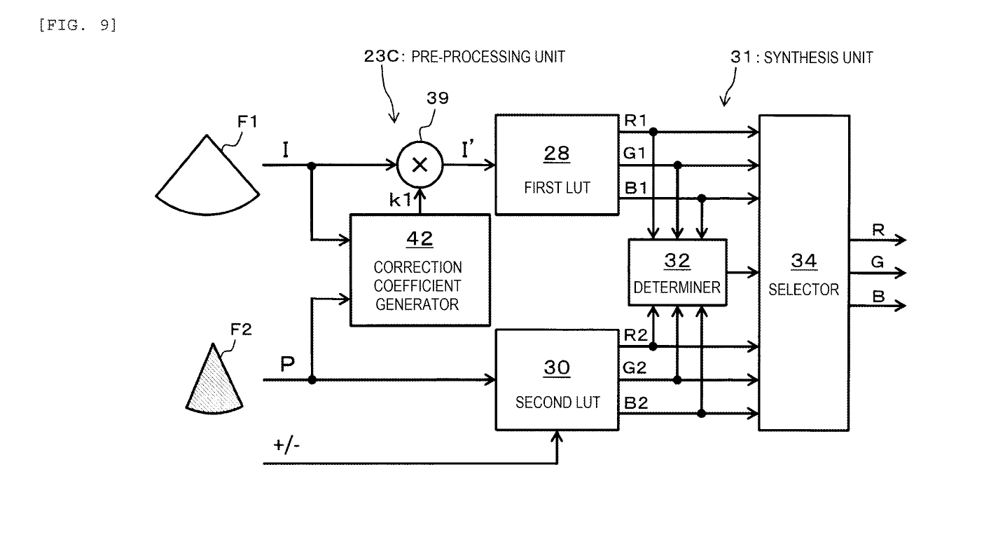

A second configuration example of the image processing unit is illustrated in FIG. 7. A display processing unit in the second configuration example includes a pre-processing unit 23A and a synthesis unit 31A. The second configuration example is equivalent to a first modification of the first configuration example illustrated in FIG. 3. Note that, the same configurations as those illustrated in FIG. 3 are denoted by the same reference numerals, and the description thereof will be omitted. The same applies to the drawings subsequent to FIG. 8.

In the second configuration example illustrated in FIG. 7, the luminance value I and the power value P′ after correction are mutually compared in the determiner 36, and any one of the luminance value I and the power value P′ after correction is selected based on the mutual comparison result. In practice, anyone of the color data set (R1, G1, B1) corresponding to the luminance value I and the color data set (R2, G2, B2) corresponding to the power value P′ after correction is selected. In such a determination method, the same operational effects can also be obtained as those of the first configuration example. For example, the determiner 36 may compare the luminance value I with a first threshold, and compare the power value P′ after correction with a second threshold, so as to determine whether to adopt the luminance value I or the power value P′ after correction based on the two comparison results (according to which pattern in four patterns it corresponds to).

A third configuration example of the image processing unit is illustrated in FIG. 8. A display processing unit in the third configuration example includes a pre-processing unit 23B and the synthesis unit 31. The third configuration example is equivalent to a second modification of the first configuration example illustrated in FIG. 3. Only the luminance value I is input to a correction coefficient generator 38, and the correction coefficient generator 38 generates a correction coefficient k based on the luminance value I. The correction coefficient k is multiplied by the power value P. In the third configuration example, the same operational effects can also be obtained as those of the first configuration example. However, according to the situation, in order to apply more appropriate pre-processing, it is desirable to obtain the correction coefficient k based on a combination of the luminance value I and the power value P as in the first configuration example.

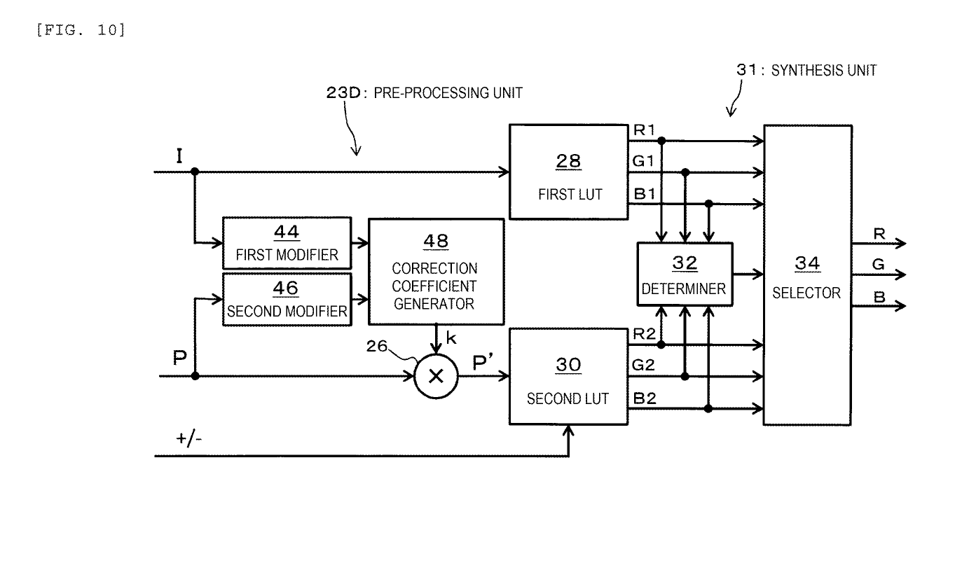

A fourth configuration example of the image processing unit is illustrated in FIG. 9. A display processing unit in the fourth configuration example includes a pre-processing unit 23C and the synthesis unit 31. In the fourth configuration example, a correction coefficient generator 42 generates a correction coefficient k1 based on the luminance value I and the power value. The correction coefficient k1 is multiplied by the luminance value I in a multiplier 39, so that a luminance value I′ after correction is obtained. The luminance value I′ after correction is input to the first LUT 28. That is, in the fourth configuration example, the luminance value I is corrected instead of the power value P, and specifically, the luminance value I is corrected by enhancement of the luminance value I. Thus, a possibility that the color data set (R1, G1, B1) corresponding to the luminance value I′ is selected in the determiner 32 is increased. However, in a case where the luminance value I is already saturated or is close thereto, it is difficult to adopt the fourth configuration example; and in a case where it is desired to maintain luminance value distribution, it is difficult to adopt the fourth configuration example. In a case where such a problem does not occur or it is desired to maintain a two-dimensional distribution of power, it is desirable to adopt the fourth configuration example.

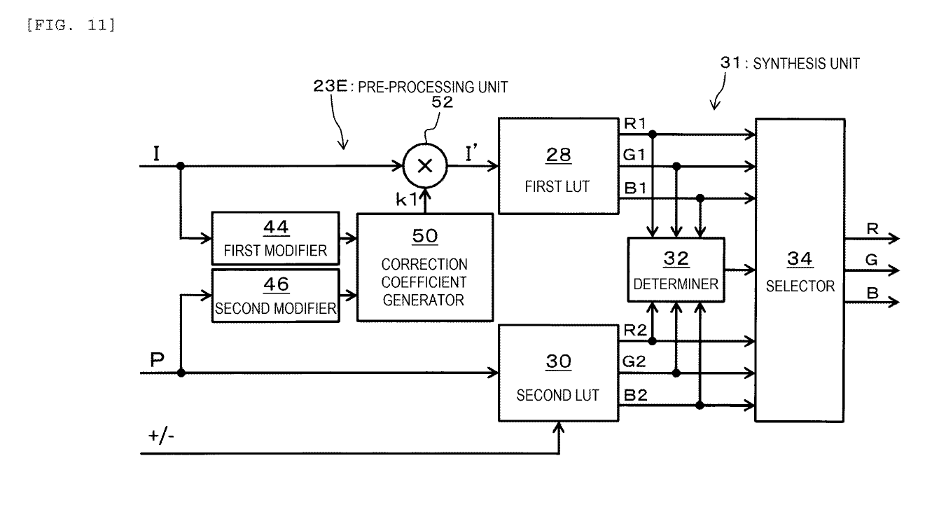

A fifth configuration example of the display processing unit is illustrated in FIG. 10. A display processing unit in the fifth configuration example includes a pre-processing unit 23D and the synthesis unit 31. In the fifth configuration example, a first modifier 44 and a second modifier 46 are provided in a front stage of a correction coefficient generator 48. The first modifier 44 modifies the luminance value I only in the generation of a correction coefficient, and the luminance value after modification is supplied to the correction coefficient generator 48. Various functions may be adopted as a modification function therefor. The second modifier 46 modifies the power value P only in the generation of the correction coefficient, and the power value after modification is supplied to the correction coefficient generator 48. Various functions may be adopted as a modification function therefor. The correction coefficient k is multiplied by the power value P.

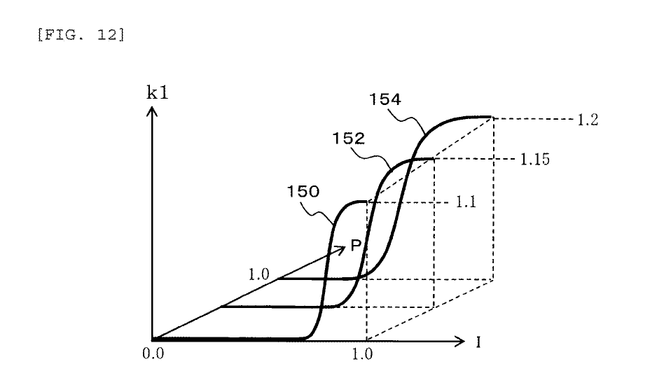

A sixth configuration example of the display processing unit is illustrated in FIG. 11. A display processing unit in the sixth configuration example includes a pre-processing unit 23E and the synthesis unit 31. In the sixth configuration example, the first modifier 44 and the second modifier 46 are provided in a front stage of a correction coefficient generator 50 as in the fifth configuration example. The correction coefficient generator 50 generates a correction coefficient k1 based on the luminance value after modification and the power value, and supplies the correction coefficient k1 to a multiplier 52. The luminance value I′ after modification multiplied by the correction coefficient k1 is supplied to the first LUT 28.

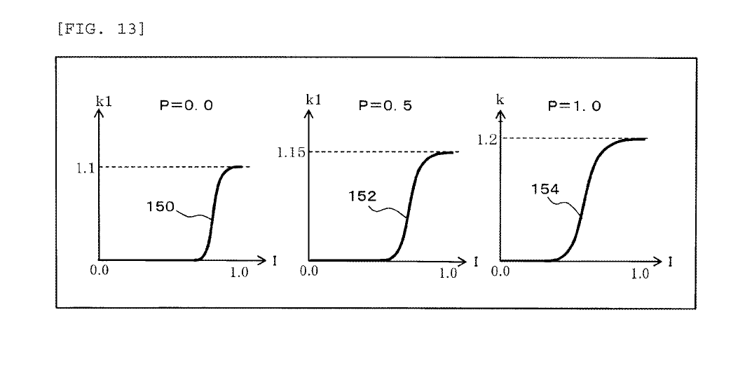

In a case where the fourth configuration example illustrated in FIG. 9 or the sixth configuration example illustrated in FIG. 11 is adopted, for example, a correction coefficient generator having a three-dimensional function illustrated in FIG. 12 may be used. In FIG. 12, a first horizontal axis represents a luminance value (however, a normalized luminance value) I. A second horizontal axis represents a power value (however, a normalized power value) P. A vertical axis represents a correction coefficient k1. In order to aid in understanding the shape of the three-dimensional function, FIG. 12 illustrates three two-dimensional functions 150, 152, and 154 corresponding to three points of a power value P=0.0, a power value P=0.5, and a power value P=1.0, and contents of which are further specifically illustrated in FIG. 13. In the illustrated example, the correction coefficient k1 gradually increases as the luminance value I increases at any of the power values P. When the power value P increases, a rising position is shifted to a lower luminance side in the two-dimensional functions 150, 152, and 154. A maximum value of the correction coefficient k1 is greater than 1.0 for enhancement of the luminance value I. As the power value P increases, a maximum value of the correction coefficient k1 gradually increases over three functions. However, the three-dimensional functions illustrated in FIGS. 12 and 13 are examples.

According to the above embodiment, in a case where a synthesized image is generated by synthesizing a monochrome tomographic image and a color power image, any one of images (particularly a power image) can be prevented from being excessively displayed. In other words, according to the above embodiment, in a case where a method of selecting any one of the two pixel values is adopted for each display coordinate, problems that easily occur in the method can be solved or alleviated while the method is maintained. In a case where a tomographic image and an image other than the power image are synthesized, and a case where an image other than the tomographic image and a power image are synthesized, the above configuration may also be adopted. The pre-processing may be selectively executed by the user, and whether or not the execution of the pre-processing is necessary may be automatically determined.

1. An ultrasonic diagnostic apparatus, comprising:
a pre-processing section that performs pre-processing on an input pixel value pair including a first input pixel value constituting a first ultrasonic image and a second input pixel value constituting a second ultrasonic image, in which a correction coefficient is generated based on at least one input pixel value in the input pixel value pair, and at least one input pixel value in the input pixel value pair is corrected based on the correction coefficient; and
a synthesis section that inputs an input pixel value pair after pre-processing, generates an output pixel value constituting a display image based on the input pixel value pair after pre-processing, and outputs the output pixel value.

2. The ultrasonic diagnostic apparatus according to claim 1, wherein
the first ultrasonic image is a tomographic image representing a cross section of a tissue,
the second ultrasonic image is a power image representing a two-dimensional distribution of power of Doppler information,
the display image is a synthesized image generated by synthesizing the tomographic image and the power image,
the first input pixel value is a luminance value corresponding to an echo value, and
the second input pixel value is a power value.

3. The ultrasonic diagnostic apparatus according to claim 2, wherein
the pre-processing section includes:
a generation section that generates the correction coefficient based on at least the luminance value; and
a correction section that corrects the power value based on the correction coefficient, and
the correction coefficient functions as a coefficient that suppresses the power value.

4. The ultrasonic diagnostic apparatus according to claim 3, wherein
the generation section generates the correction coefficient based on a combination of the luminance value and the power value.

5. The ultrasonic diagnostic apparatus according to claim 3, wherein
the synthesis section is a section that selects any one of the luminance value and the power value after correction based on a mutual comparison between the luminance value and the power value after correction, and
the luminance value tends to be selected as a result of the mutual comparison when the power value is suppressed, and in a case where the power value after correction is selected as the output pixel value, the output pixel value is suppressed.

6. An image processing method, comprising:
a pre-processing step of performing pre-processing on an input pixel value pair including a first input pixel value constituting a first ultrasonic image and a second input pixel value constituting a second ultrasonic image, that is, correcting at least one input pixel value in the input pixel value pair based on at least the other input pixel value in the input pixel value pair; and
a selection step of inputting the input pixel value pair after pre-processing, selecting any one of the input pixel values based on mutual comparison of the input pixel value pair after pre-processing, and outputting the selected input pixel value as an output pixel value.

7. The image processing method according to claim 6, wherein
the first ultrasonic image is a monochrome tomographic image representing a tissue,
the second ultrasonic image is a color power image representing blood flow, and
a power value which is the second input pixel value is corrected before the selection step is executed.
